(19) United States
(12) Patent Application Publication
(10) Pub. No.: US 2022/0054082 A1
(43) Pub. Date: Feb. 24, 2022

(54) SKIN MARKING PATCH

(71) Applicant: Buddi Limited, Rickmansworth (Hertfordshire) (GB)

(72) Inventors: Sara MURRAY, Rickmansworth (GB); Rowena MURRAY, Rickmansworth (GB)

(21) Appl. No.: 17/405,968

(22) Filed: Aug. 18, 2021

Related U.S. Application Data
(60) Provisional application No. 63/068,006, filed on Aug. 20, 2020.

(51) Int. Cl. A61B 5/00; A61B 5/145

(52) U.S. Cl. CPC A61B 5/4845; A61B 2560/0412; A61B 5/14517; A61B 5/6833

(57) ABSTRACT

A skin patch for use on a skin portion of a subject. The skin patch includes an analyte detector that detects an analyte from perspiration. The skin patch is adapted to release a marking agent such as a dye to mark the skin portion upon detection of the analyte.

### CROSS REFERENCE TO RELATED APPLICATION

The present application claims priority to U.S. Provisional Patent Application No. 63/068,006, filed Aug. 20, 2020, which is hereby incorporated by reference in its entirety.

### FIELD OF THE DISCLOSURE

The present disclosure relates to a skin patch adapted to release a dye upon detection of a substance of interest present in the sweat of a subject to mark the skin.

### BACKGROUND

Monitoring of alcohol and drug consumption comes into both the sphere of law enforcement and medicine. In law enforcement offenders may have as part of their sentence an abstinence regime put in place, so as they avoid substances that are known to trigger undesirable behaviours. In medicine the verification of abstinence from an addictive substance has always proved difficult, especially when the patient still has some degree of control over their addictive behaviour.

In offender management regimes there is a new trend toward abstinence orders. The current state of the art in real-time alcohol monitoring involves the user being prompted to blow into an image-secured breathalyser or a less accurate body mounted fuel-cell device (the same technology as modern breathalysers) which detects evaporating alcohol from the skin. Both these devices are intended for offender management applications and cannot realistically be seen as a solution for those who want to stay sober under other circumstances. The current state of the art in non-invasive drug monitoring is an adhesive patch containing an absorbent paper that on removal undergoes laboratory examination by gas-chromatography and mass-spectroscopy for traces of known drugs or its metabolites.

Patent documents U.S. Pat. No. 5,220,919A and U.S. Pat. No. 7,930,927B2 describe examples of trans-dermal fuel-cell based alcohol monitors. US 20150212063 describes an image user-verification breathalyser.

Several transdermal sweat collection patches have been reported. WO98/14768 describes a transdermal chemical monitoring provided with a membrane structure and an analyte capture material. The analysis of the captured analyte is left undescribed and remote from the patch and its chemical processes. An alternative passive absorber solution is described in U.S. Pat. No. 4,756,314A and U.S. Pat. No. 5,638,815, with the reference therein providing further solutions based on reservoir material based collection systems. U.S. Pat. No. 6,585,646 teaches of a method for analysing the collected samples.

The general aim is to determine whether a subject has taken a substance within a given time-period in a verifiable manner. The test should not suffer from interferences or false positives that may arise from other individual consuming the substance in the vicinity of the subject wearing the device. In addition, the test should be easily carried out and the cost per test should be relatively low.

Existing solutions address the general problem and the need to identify the test with the wearer but remain relatively expensive. It is an object of the disclosure to address one or more of the above mentioned limitations.

### SUMMARY OF THE DISCLOSURE

According to a first aspect of the disclosure, there is provided a skin patch for use on a skin portion of a subject, the skin patch comprising an analyte detector adapted to detect an analyte from perspiration, the skin patch being adapted to release a marking agent to mark the skin portion upon detection of the analyte.

Optionally, the analyte detector comprises an assay adapted to detect the analyte.

Optionally, the assay is an antibody-based assay or an enzyme-based assay.

Optionally, the assay comprises a plurality of dye molecules bound to a support structure, and wherein upon detection of the analyte the dye molecules detach from the support structure.

Optionally, the support structure comprises a substrate or a gel. For instance, the support structure may be a polymer matrix, a gel matrix, or a polymer layer.

Optionally, the support structure is functionalised.

Optionally, the support structure comprises a plurality of probes adapted to interact with the analyte such that a property of the support structure changes upon probe-analyte interaction to release the plurality of dye molecules. For instance, the property may be an electrical property.

Optionally, the probe-analyte interaction includes at least one of an enzymatic reaction and an antibody binding event.

Optionally, the marking agent is held in a container, and upon detection of the analyte the analyte detector produces a chemical product to degrade the container and release the marking agent. For instance, the container may be formed of a membrane that degrades in the presence of the chemical product.

Optionally, the analyte detector is provided between an inner membrane and an outer membrane, wherein the inner membrane is permeable to perspiration.

Optionally, the inner membrane is adapted to provide adhesion to the skin region.

Optionally, the inner membrane is partly perforated so that the inner membrane tears upon interference with the patch.

Optionally, the analyte includes ethanol.

Optionally, the marking agent comprises a dye.

Optionally, the skin patch comprises an identifier.

Optionally, the skin patch comprises a plurality of assays, each assay being adapted to identify a different analyte, the skin patch being adapted to mark the skin region with a pattern corresponding to the one or more detected analytes.

### DETAILED DESCRIPTION

The disclosure is described in further detail below by way of example and with reference to the accompanying drawings, in which:

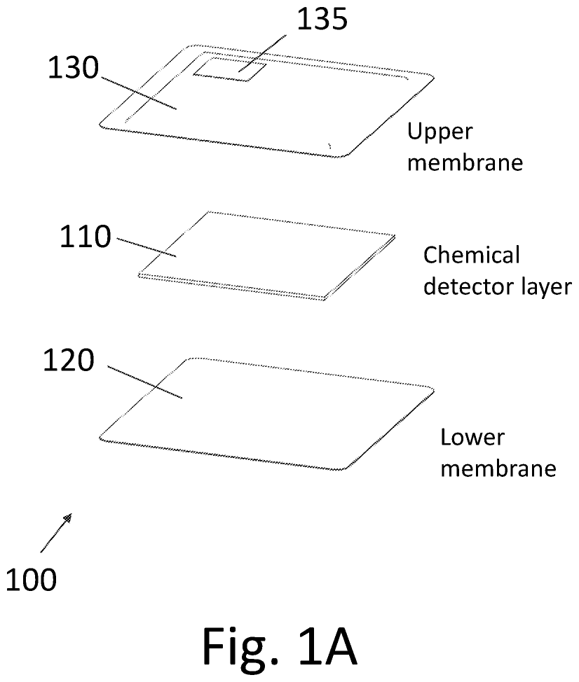

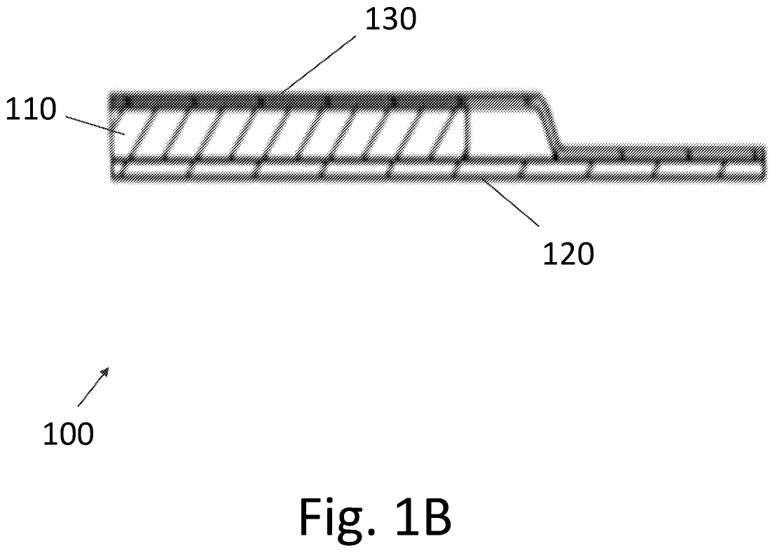

FIG. 1A is an exploded view of a skin patch;

FIG. 1B is a profile cross section of the skin patch of FIG. 1A;

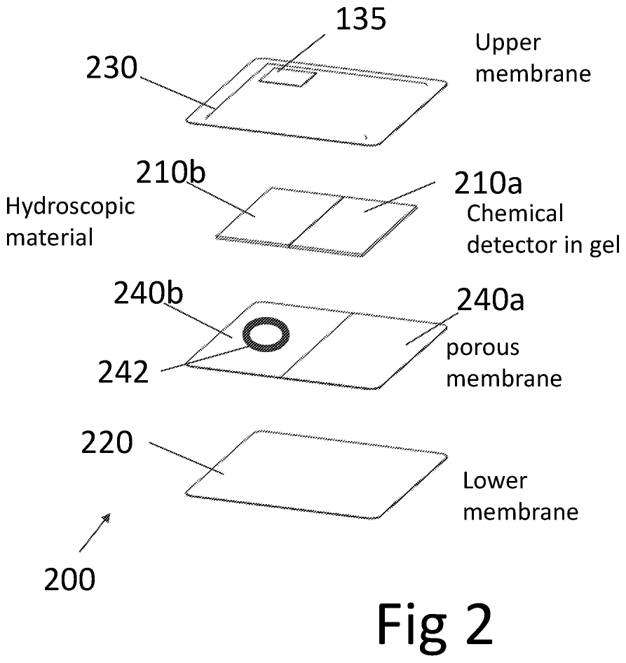

FIG. 2 is an exploded view of a skin patch provided with a hydroscopic material;

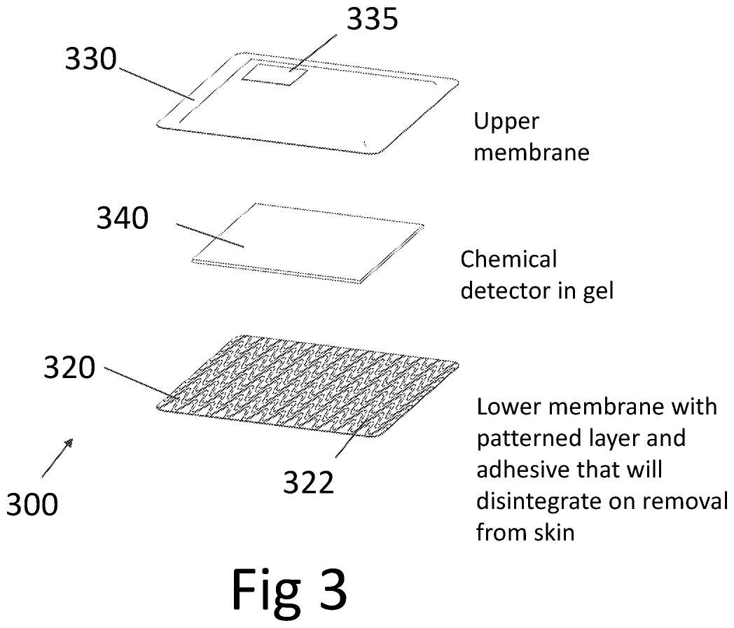

FIG. 3 is an exploded view of a skin patch provided with a patterned lower membrane;

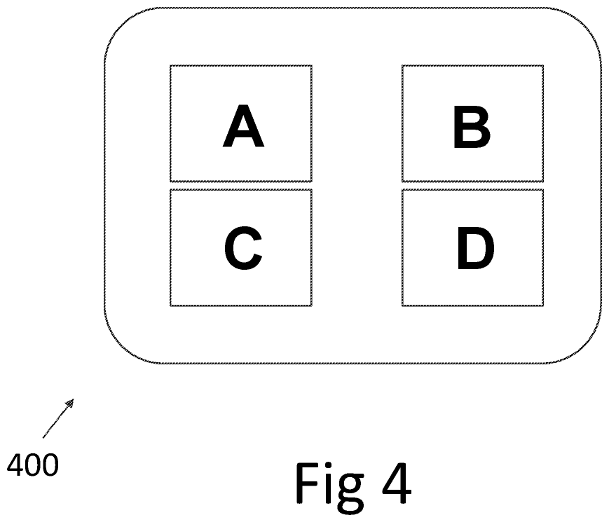

FIG. 4 is a marker for use with the skin patch.

FIG. 1A illustrates a skin patch 100 for detecting a substance ingested by the subject wearing the patch and present in the sweat of the subject. FIG. 1B shows a profile cross section of the skin patch 100.

The skin patch 100 includes an analyte detector adapted to detect an analyte from perspiration. In this example the analyte detector is provided by a chemical detector layer 110, also referred to as active layer, sandwiched between first membrane 120 and a second membrane 130. The skin patch 100 is also adapted to release a marking agent to mark the skin portion upon detection of the analyte.

The chemical detector layer 110 may be made of a gel or a polymer containing a chemical detection and marker release system, and an exterior membrane capable of passing water to the outside of the gel or polymer. The chemically active layer 110 can use a high-specificity analytical mechanism to generate a signal also refer to as dying signal to release a dye for marking a skin portion of the wearer.

The first membrane 120 is attachable to a skin portion of the subject and referred to as the lower or inner membrane. For example, the lower membrane 120 may have an adhesive layer. The membrane 120 is permeable and capable of passing all the components of sweat emanating from the wearer skin, including both water and lipid soluble compounds towards the chemical detector layer 110. For example, the lower membrane 120 may include a high porosity membrane such as a paper membrane. A pattern of adhesive may be printed on the side of the membrane facing the skin of the subject. The combination of the membrane and patterned adhesive may be made such that on removal the lower membrane 120 is destroyed in a manner that prevents reapplication of the patch. The membrane 120 may be designed to filter out various interferants generated by the skin cells, from the chemical detector layer 110.

The second membrane 130, also referred to as upper or outer membrane is provided with an identifier 135. For instance, the identifier 135 may be a 2D barcode. The upper membrane 130 is selected or designed to be impermeable to the substance of interest, hence preventing ingress of that substance from the outer environment into the chemically active layer 110. Additional protective layers may be used to block the substance of interest and prevent it from entering the chemically active layer 110 from any direction other than through the lower membrane 120 which is in contact with the skin.

The upper membrane 130 may be selected or designed to prevent the dying agent passing from the chemically active layer 110 to the outside. As a result the concentration of dying agent present in the chemically active layer 110 can be maintained and dying of the subject cloth can be prevented. Several commercially available membranes made from species of polyurethane and polyethylene allow the transport of water whilst preventing or limiting the transmission of other substances.

The patch 100 may be designed to be removable or peeled in two separate portions, for instance a first portion including the identifier 135 and a second portion including the chemical detector layer. In this way, it is possible to remove the second portion first to reveal the markable skin region and take a picture that would show both the identifier 135 and the skin region that may be marked.

In operation, when the substance of interest enters the chemically active layer 110 a dying agent present in the layer 110 is released through the lower membrane 120 to form a semi-permanent mark on the wearer's skin. Once the patch has been removed, an image of the skin can be taken, hence identifying that the wearer has exposed the patch to the substance of interest.

The chemically active layer 110 and the dye release mechanism enabling to dye the skin of the subject upon detection of a substance of interest can be implemented in various ways.

In a first approach the dye is provided in a solution or a gel. Upon detection of the substance of interest, the dye is released onto the skin through the lower membrane 120.

The chemical active layer 110 may include a biological assay designed to detect the substance of interest.

Biological assays may use antibodies and/or enzymes to identify specific proteins and analytes of interest. Antibodies may be used to detect relatively large or complex molecules in which several binding sites are present. For instance antibodies can be used to bind to opiates and their metabolites. Enzymes may be used to detect smaller molecules, for example alcohol may be detected using an enzyme.

In a conventional bioassay a staining procedure is used to fix a dye to a binding site in a stationary sample. For example, an antibody connected to a dye molecule may be placed in solution before being added to an immobilised sample. Once the antibody has bound to the sites of interest in the sample, the rest of the solution is washed off and the sample is examined for detecting presence of the dye.

In contrast the bioassay used in the active layer 110 is designed to release the dye upon probe/target binding.

In an exemplary embodiment the active layer 110 includes a plurality of dye molecules bound to a support structure. The support structure may be a substrate such as a solid surface layer or an inert molecular structure such as a gel. Dye molecules are bound to the substrate or to the inert molecular structure of the active layer. The nature of the binding is designed such that the bound is broken and the dye released in the presence of the substance of interest.

The support structure may be functionalised with a plurality of probe molecules adapted to interact with the analyte for example through a binding process. A property of the support structure, for instance an electrical property, may change upon probe-analyte interaction to release the dye molecules. As explained above the probe molecule may be a binding antibody.

When the analyte of interest, also referred to as target molecule, enters the active layer 110 it remains mobile in the solution or gel until it binds to the probe molecule. The interaction between the target and the probe molecules results in the release of the dye molecules from their binding sites which are then free to enter the solution. The dye can then migrate through the lower membrane 120 onto the user's skin and leave a semi-permanent mark.

Alternatively an enzyme mechanism may be used in which the dye is activated or released by the presence of the products of the enzymatic reaction. For instance, the dye may be released by the presence of peroxide (a common product of an oxidase enzyme) in concentrations above a certain level.

In a second approach the marking agent is held in a container. The container may be formed of a membrane that degrades in the presence of the chemical product. Upon detection of the analyte the analyte detector produces a chemical product to degrade the container and release the marking agent.

For instance, the active layer 110 may be provided with a vessel or dye-membrane that contains the dye. Upon detection of the substance of interest, a chemical product is produced which opens the vessel by disintegrating at least part of the dye-membrane. Once released the dye migrates to the wearer's skin via the lower membrane 120, where it then binds to the stratum corneum to give a semi-permanent mark.

The product may be generated as a chemical product of an antibody binding process or an enzyme reaction. For instance, a chemical process may be chosen such that the chemical product is released by the analyte.

Optionally, the active layer 110 may include an electronic component adapted to generate an electrical potential. In this case, the electrical potential may be used to improve both the enzymatic detection efficiency and the dye release mechanism.

In a third approach the bioassay is designed to be exposed to the wearer skin. In this example, the active layer 110 may include an antibody/dye combination that forms a dry layer. In use the perspiration would moisten the dry layer, hence releasing antibody-dye conjugates that may come into contact with the wearer's stratum corneum and free to move in solution (wearer's perspiration). In some embodiments the active layer 110 may be in direct contact with the skin, that is without provision of the lower membrane. In other embodiments the lower membrane is provided between the active layer 110 and the skin.

If a substance of interest is present in the wearer's skin, then the antibody will bind to it. To detect the eventual presence of the substance of interest, the patch would be removed and the area below it washed and assessed for dye content.

In a fourth approach the bioassay uses the action of an antibody or an enzyme to detect the substance of interest with Green Fluorescent Protein (GFP)or other fluorescent dyes. In this case the fluorescent dye would go into its fluorescent state in the presence of the substance of interest.

FIG. 2 shows an exploded view of a skin patch 200 provided with a hydroscopic material for drawing sweat through the chemical detector.

The skin patch 200 includes a layer 210 formed of a chemical detector 210a adjacent to a hydroscopic material 210b . The hydroscopic material 210b is used to attract the sweat through to the chemical detector 210a . For example, the hydroscopic material 210b may be made, at least in part, of silica gel or bentonite clay.

A layer of differential porosity 240 may be provided between the layer 210 and the lower membrane 220. The layer 240 has a porous part 240a adjacent to an impervious part 240b also referred as non-porous part. The layer 240 is used to direct the sweat through the chemical detector 210a and thence to the hydroscopic material 210b . The impervious part 240b may be provided with a slit 242 for allowing the marking agent to reach the skin through the lower membrane 220. The slit may have a distinctive shape, for instance a letter shape or a geometrical shape such as a circular or a triangular shape.

In used the sweat is drawn from the skin of the subject towards the hydroscopic material 210b through the porous part 240a and the detector 210a . The impervious part 240b concentrates the marking agent/dye and let it leach into the skin through the slit 242 via the lower membrane 220. In FIG. 2, the hydroscopic material 210b is located adjacent to the detector 210a . However, in an alternative embodiment the hydroscopic material 210b may be located above the detector 210a . In this case the membrane 240 is not necessary and may be removed.

FIG. 3 shows a perspective view of a skin patch 300 comprising a tamper detection feature. In this case, the lower membrane 320 has a plurality of weakening lines 322. These features allow the lower membrane to partially tear and/or delaminate on removal from the skin. Alternatively, an adhesive film of the inner layer may be patterned to provide different pulling forces across the lower membrane, such that the lower membrane is structurally deformed upon its removal from the skin.

The skin patch as described with reference to FIG. 1, 2 or 3 may be adapted to identify multiple analytes. The skin patch may include a plurality of assays, each assay being adapted to identify a different analyte. The skin patch may further be adapted to mark the skin region with a pattern corresponding to the one or more detected analytes. For instance skin markings features may be provided to indicate the detection of the different analytes. The marking can be differentiated by a combination of dye/ink type, location with respect to each other and marking shapes. The patterning for the marking shapes can be made in a patched glue layer, with a suitable border stopping the self-destructing glue smudging the pattern.

FIG. 4 illustrates a marker for use with the skin patch. The marker 400 may be implemented as part of the lower membrane and/or the chemical detector layer. The marker 400 includes four regions for identifying different analytes with a distinct shape, in this cases four letters each letter corresponding to a different analyte. Each shape may form a vessel or template in the lower membrane of the patch, that contains a marking agent/dye. The marker shapes may be made as a mask in the glue layer. Upon identification of the analyte of interest the glue pattern becomes interrupted in the zone of the marker, hence marking the skin of the subject.

It will be appreciated that only non-toxic dye types should be considered. Ideally the dye, or ink, should leave a mark that is visible to the human eye. Alternatively the dye, or ink, can contain a fluorescent agent which can only be observed under UV light. The dye or ink should persist in the wearer's stratum corneum for a pre-defined measurement period. The measurement period may vary depending on the application. For instance, the period may last for one week or one month.

A skilled person will therefore appreciate that variations of the disclosed arrangements are possible without departing from the disclosure.

Accordingly, the above description of the specific embodiments is made by way of example only and not for the purposes of limitation. It will be clear to the skilled person that minor modifications may be made without significant changes to the operation described.

1. A skin patch for use on a skin portion of a subject, the skin patch comprising an analyte detector adapted to detect an analyte from perspiration, the skin patch being adapted to release a marking agent to mark the skin portion upon detection of the analyte.

2. The skin patch as claimed in claim 1, wherein the analyte detector comprises an assay adapted to detect the analyte.

3. The skin patch as claimed in claim 2, wherein the assay is an antibody-based assay or an enzyme-based assay.

4. The skin patch as claimed in claim 2, wherein the assay comprises a plurality of dye molecules bound to a support structure, and wherein upon detection of the analyte the dye molecules detach from the support structure.

5. The skin patch as claimed in claim 4, wherein the support structure comprises a substrate or a gel.

6. The skin patch as claimed in claim 4, wherein the support structure is functionalised.

7. The skin patch as claimed in claim 4, wherein the support structure comprises a plurality of probes adapted to interact with the analyte such that a property of the support structure changes upon probe-analyte interaction to release the plurality of dye molecules.

8. The skin patch as claimed in claim 7, wherein the probe-analyte interaction includes at least one of an enzymatic reaction and an antibody binding event.

9. The skin patch as claimed in claim 1, wherein the marking agent is held in a container, and wherein upon detection of the analyte the analyte detector produces a chemical product to degrade the container and release the marking agent.

10. The skin patch as claimed in claim 1, wherein the analyte detector is provided between an inner membrane and an outer membrane, wherein the inner membrane is permeable to perspiration.

11. The skin patch as claimed in claim 10, wherein the inner membrane is adapted to provide adhesion to the skin region.

12. The skin patch as claimed in 11, wherein the inner membrane is partly perforated so that the inner membrane tears upon interference with the patch.

13. The skin patch as claimed in claim 1, wherein the analyte includes ethanol.

14. The skin patch as claimed in claim 1 wherein the marking agent comprises a dye.

15. The skin patch as claimed in claim 1, further comprising an identifier.

16. The skin patch as claimed in claim 2, comprising a plurality of assays, each assay being adapted to identify a different analyte, the skin patch being adapted to mark the skin region with a pattern corresponding to the one or more detected analytes.
